(19) United States
(12) Patent Application Publication
(10) Pub. No.: US 2024/0138863 A1
(43) Pub. Date: May 2, 2024

(54) MECHANICAL THROMBECTOMY SYSTEMS

(71) Applicant: Boston Scientific Scimed, Inc., Maple Grove, MN (US)

(72) Inventors: Ewnet Gebrehiwot, Eden Prairie, MN (US); Gary John Pederson, Jr., Albertville, MN (US); Michael Nagel, Andover, MN (US)

(73) Assignee: Boston Scientific Scimed, Inc., Maple Grove, MN (US)

(21) Appl. No.: 18/384,432

(22) Filed: Oct. 27, 2023

Related U.S. Application Data
(60) Provisional application No. 63/420,046, filed on Oct. 27, 2022.

(51) Int. Cl. A61B 17/22; A61B 17/221

(52) U.S. Cl. CPC A61B 17/22031; A61B 17/221; A61B 2017/00336

(57) ABSTRACT

An illustrative mechanical thrombectomy system may comprise an outer catheter extending from a proximal end to a distal end and defining a first lumen extending therethrough and an inner sheath extending from a proximal end to a distal end and defining a second lumen extending therethrough. The inner sheath may be slidably disposed within the first lumen of the outer catheter. An inner shaft may be slidably disposed within the second lumen of the inner sheath and an expandable anchor mechanism may be coupled to the distal end region of the inner shaft. The expandable anchor mechanism may be configured to move between a collapsed delivery configuration and an expanded configuration.

### CROSS-REFERENCE TO RELATED APPLICATIONS

This application claims the benefit of priority under 35 U.S.C. § 119 of U.S. Provisional Application No. 63/420,046, filed Oct. 27, 2022, the entire disclosure of which is hereby incorporated by reference.

### TECHNICAL FIELD

The present disclosure pertains to medical devices and methods for making and using medical devices. More particularly, the present disclosure pertains to mechanical thrombectomy systems.

### BACKGROUND

A wide variety of intracorporeal medical devices have been developed for medical use, for example, intravascular use. Some of these devices include mechanical thrombectomy systems. For example, a mechanical thrombectomy device may remove thrombotic material from the body without the use of a lytic agent. These devices are manufactured by any one of a variety of different manufacturing methods and may be used according to any one of a variety of methods. Of the known clot mechanical thrombectomy systems and methods for making and using the same, each has certain advantages and disadvantages. There is an ongoing need to provide alternative mechanical thrombectomy systems as well as alternative methods for making and using mechanical thrombectomy systems.

### BRIEF SUMMARY

The disclosure provides design, material, manufacturing method, and use alternatives for clot entrapment devices and/or systems and methods for making and using the same.

In a first example, a mechanical thrombectomy system may comprise an outer catheter extending from a proximal end to a distal end and defining a first lumen extending therethrough, an inner sheath extending from a proximal end to a distal end and defining a second lumen extending therethrough, the inner sheath slidably disposed within the first lumen of the outer catheter, an inner shaft extending from a proximal end to a distal end region, the inner shaft slidably disposed within the second lumen of the inner sheath, an expandable anchor mechanism coupled to the distal end region of the inner shaft, the expandable anchor mechanism configured to move between a collapsed configuration and an expanded configuration, and a handle coupled to the proximal end of the outer catheter.

Alternatively or additionally to any of the examples above, in another example, the anchor mechanism may comprise a torsion spring.

Alternatively or additionally to any of the examples above, in another example, the torsion spring may comprise a helically wound portion and one or more free ends.

Alternatively or additionally to any of the examples above, in another example, the helically wound portion may bias the one or more free ends into an expanded configuration.

Alternatively or additionally to any of the examples above, in another example, a biasing force may be configured to radially deflect the one or more free ends radially inwards.

Alternatively or additionally to any of the examples above, in another example, the mechanical thrombectomy system may further comprise a polymer jacket disposed over at least a portion of the one or more free ends.

Alternatively or additionally to any of the examples above, in another example, the anchor mechanism may comprise an expandable basket.

Alternatively or additionally to any of the examples above, in another example, the expandable basket may be self-expanding.

Alternatively or additionally to any of the examples above, in another example, the mechanical thrombectomy system may further comprise a central wire coupled to a distal end of the expandable basket.

Alternatively or additionally to any of the examples above, in another example, the mechanical thrombectomy system may further comprise an outer sheath extending from a proximal end to a distal end, the outer sheath disposed over the outer catheter.

Alternatively or additionally to any of the examples above, in another example, the outer catheter may further comprise an expandable distal end region adjacent to the distal end, the expandable distal end region may be movable between a collapsed configuration and an expanded configuration.

Alternatively or additionally to any of the examples above, in another example, the expandable distal end region may comprise two or more support members and a membrane extending between the two or more support members.

Alternatively or additionally to any of the examples above, in another example, when the outer sheath is disposed over the expandable distal end region, the expandable distal end region may be biased into the collapsed configuration and when the outer sheath is proximally retracted relative to the outer catheter, the expandable distal end region may expand into the expanded configuration.

Alternatively or additionally to any of the examples above, in another example, when the inner sheath is disposed over the anchor mechanism, the anchor mechanism may be biased into the collapsed configuration and when the inner sheath is proximally retracted relative to the anchor mechanism, the anchor mechanism may expand into the expanded configuration.

Alternatively or additionally to any of the examples above, in another example, when the anchor mechanism is in the expanded configuration, distal advancement of the inner sheath may bias the anchor mechanism to the collapsed configuration.

In another example, a mechanical thrombectomy system may comprise an outer sheath extending from a proximal end to a distal end and defining a first lumen extending therethrough, an intermediate sheath extending from a proximal end to a distal end and defining a second lumen extending therethrough, the intermediate sheath slidably disposed within the first lumen of the outer sheath, an inner sheath extending from a proximal end to a distal end and defining a third lumen extending therethrough, the inner sheath slidably disposed within the second lumen of the intermediate sheath, an inner shaft extending from a proximal end to a distal end region, the inner shaft slidably disposed within the second lumen of the inner sheath, and an expandable anchor mechanism coupled to the distal end region of the inner shaft, the expandable anchor mechanism configured to move between a collapsed delivery configuration and an expanded configuration, the anchor mechanism comprising a torsion spring.

Alternatively or additionally to any of the examples above, in another example, the intermediate sheath may further comprise an expandable distal end region adjacent to the distal end, the expandable distal end region movable between a collapsed configuration and an expanded configuration.

Alternatively or additionally to any of the examples above, in another example, the expandable distal end region may comprise two or more support members and a membrane extending between the two or more support members.

Alternatively or additionally to any of the examples above, in another example, when the outer sheath is disposed over the expandable distal end region, the expandable distal end region may be biased into the collapsed configuration and when the outer sheath is proximally retracted relative to the intermediate sheath, the expandable distal end region may expand into the expanded configuration.

In another example, a method for removing a clot may comprise advancing a mechanical thrombectomy system through a vasculature to a target location. The mechanical thrombectomy system may comprise an outer catheter extending from a proximal end to a distal end and defining a first lumen extending therethrough, an inner sheath extending from a proximal end to a distal end and defining a second lumen extending therethrough, the inner sheath slidably disposed within the first lumen of the outer catheter, an inner shaft extending from a proximal end to a distal end region, the inner shaft slidably disposed within the second lumen of the inner sheath, and an expandable anchor mechanism coupled to the distal end region of the inner shaft, the expandable anchor mechanism configured to move between a collapsed delivery configuration and an expanded configuration. The method may further comprise distally advancing the inner sheath and the inner shaft through a clot in the vasculature, proximally retracting the inner sheath to expand the anchor mechanism, distally advancing the outer catheter through the clot, and distally advancing the inner sheath or the outer catheter to recapture the anchor mechanism.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments of the disclosure in connection with the accompanying drawings, in which:

FIG. 1 is a schematic side view of an example clot entrapment system;

FIG. 2 is a schematic side view of a distal end region of the example mechanical thrombectomy system of FIG. 1;

FIG. 3 is a perspective view of an illustrative anchor;

FIGS. 4-8 are schematic side views of a portion of the example mechanical thrombectomy system of FIG. 1 in use within a body lumen;

FIG. 9 is a schematic side view of a portion of another example mechanical thrombectomy system disposed within a body lumen; and

FIGS. 10-11 are schematic side views of a portion of another example mechanical thrombectomy system in use within a body lumen.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

The term “distal” refers to a portion farthest away from a user when introducing a device into a patient. By contrast, the term “proximal” refers to a portion closest to the user when placing the device into the patient. Relative terms such as “proximal”, “distal”, “advance”, “retract”, variants thereof, and the like, may generally be considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user.

As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

All numeric values are herein assumed to be modified by the term “about,” whether or not explicitly indicated. The recitation of numerical ranges by endpoints includes all numbers within that range (e.g. 1 to 5 includes 1, 1.5, 2, 2.75, 3, 3.80, 4, and 5).

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the disclosure.

A mechanical thrombectomy device may remove thrombotic material from the body without the use of a lytic agent. In some cases, it may be desirable to mechanically remove sub-acute and chronic clots that contain high amounts of collagen and fibrin. Traditionally sub-acute and chronic clots may require invasive surgical intervention. The present disclosure is directed towards mechanical thrombectomy devices and methods for using the mechanical thrombectomy devices to remove any type of clot, including newer, acute thrombotic material along with older, sub-acute and chronic material. However, the devices and methods can be used to treat other conditions as desired, including, but not limited to, pulmonary embolisms.

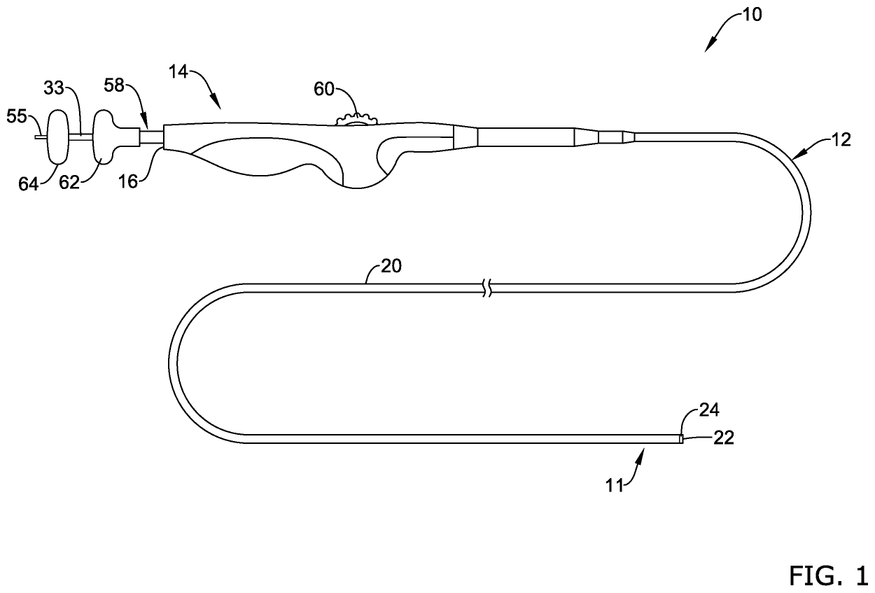

FIG. 1 depicts a schematic side view of an illustrative clot entrapment system or mechanical thrombectomy system 10. The mechanical thrombectomy system 10 may include an elongate shaft 12 and a handle 14 coupled to the elongate shaft 12. In general, the mechanical thrombectomy system 10 may be used to entrap any type of clot, including newer, acute thrombotic material along with older, sub-acute and chronic material, or the like, within a body lumen of a patient. The body lumen may be a blood vessel located near the heart (e.g., within or near a cardiac vessel), within a peripheral vessel, within a neurological vessel, or at any other suitable location. In some cases, the mechanical thrombectomy system 10 may include one or more distal anchors configured to anchor a distal end region of the mechanical thrombectomy system 10 while the clot is being removed.

The elongate shaft 12 may include a number of elongate structures that are co-axially arranged (e.g., sharing a common longitudinal axis) or that are at least partially disposed one inside of another and not necessarily sharing a common longitudinal axis. In some cases, one or more of the elongate structures may be radially offset from a central longitudinal axis of the mechanical thrombectomy system 10. The elongate shaft 12 may include an outer catheter 20 movably coupled to the handle 14. The outer catheter 20 may extend distally from a proximal end disposed within the handle 14 to a distal end 22 configured to be advanced within a body lumen. In some embodiments, the outer catheter 20 may be a tubular polymer, may be made up of a coil or braid reinforced polymer, or may be a polymer encapsulated laser cut metallic tube. For example, the outer catheter 20 may be a laser cut stainless steel tube with a polymer jacket (e.g., an inner and/or outer layer of polymer) configured to provide strength, pushability, and/or compression resistance. In some embodiments, a radiopaque marker band 24 may be positioned at or adjacent to the distal end 22. The marker band 24 may allow the distal end 22 of the outer catheter 20 to be fluoroscopically visualized during advancement of the mechanical thrombectomy system 10. It is contemplated that the marker band 24 may increase the pushability of the outer catheter 20 through a lesion such as, but not limited to, acute thrombotic material, sub-acute thrombotic material, and chronic material. The outer catheter 20 may be provided with or without a lubricious liner, such as, but not limited to, polytetrafluoroethylene (PTFE) or perfluoroalkoxy alkane (PFA). An exterior of the outer catheter 20 may include a lubricious additive. Polymers for the outer catheter 20 may include, but are not limited to, PEBAX®, urethane, polyimide, polyamide, a combination thereof, or the like.

The proximal end region of the outer catheter 20 may be attached to a gear rack assembly disposed within the handle 14 with a fastener or clip (not explicitly shown). An illustrative gear rack assembly is described in commonly assigned U.S. Patent Application 63/359,409, titled STENT DELIVERY SYSTEM, the disclosure of which is hereby incorporated by reference. As the outer catheter 20 is coupled to the gear rack assembly, proximal movement of the gear rack assembly may result in analogous proximal movement of the outer catheter 20 and distal movement of the gear rack assembly may result in analogous distal movement of the outer catheter 20. The gear rack assembly may include a plurality of teeth or gears (not explicitly shown). In practice, the teeth may be configured to engage with corresponding teeth or gears on a thumbwheel 60 of the handle. Consequently, rotation of the thumbwheel 60, via gearing thereof with the gears of the gear rack assembly, can be utilized to proximally retract and/or distally advance the gear rack assembly and, thus, the outer catheter 20. Other structural arrangements may be utilized to accomplish proximal retraction and/or distal advancement of the gear rack assembly through the actuation of the thumbwheel 60 or any other suitable actuation member.

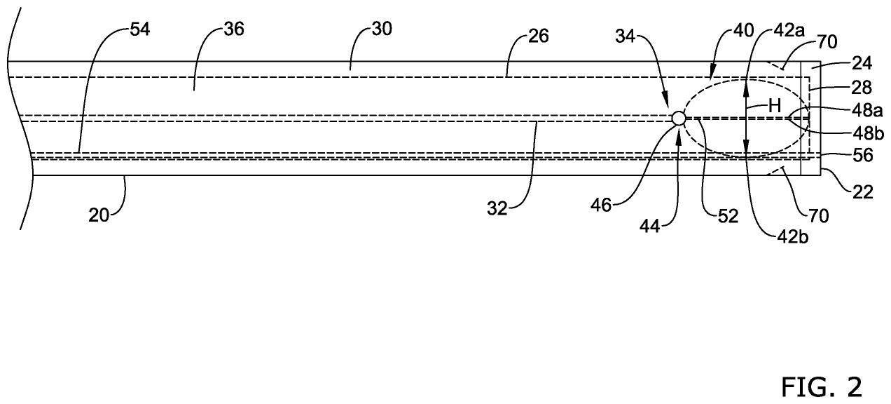

Referring additionally to FIG. 2, which illustrates a schematic side view of a distal end region 11 of the mechanical thrombectomy system 10, the outer catheter 20 may optionally include one or more cutting members 70. The cutting member 70 may extend radially inward from an inner surface of the outer catheter 20. In some cases, the cutting member 70 may be a circular blade extending about an entire inner circumference of the outer catheter 20. In other embodiments, the cutting member 70 may extend about less than entire circumference of the outer catheter 20. In yet other embodiments, the cutting member 70 may include two or more cutting members uniformly or eccentrically positioned about an inner surface of the outer catheter 20. When so provided, the cutting member 70 may help cut through the clot. The cutting member 70 may be positioned proximal to the distal end 22 of the outer catheter 20. This may help prevent or reduce damage to the blood vessel during advancement of the mechanical thrombectomy system 10. However, this is not required. In some cases, the cutting member 70 may be positioned at or distal to the distal end 22.

The elongate shaft 12 may also include an inner sheath 26 extending from a proximal end region 58 disposed proximal to a proximal end 16 of the handle 14 and through the handle to a distal end 28. The inner sheath 26 may be positioned radially inwards of the outer catheter 20 and slidably disposed within a lumen 30 thereof. For example, the inner sheath 26 may be configured to be axially and/or rotationally displaced relative to the outer catheter 20. In some embodiments, the inner sheath 26 may be a tubular polymer, may be made up of a coil or braid reinforced polymer, or may be a polymer encapsulated laser cut metallic tube. For example, the inner sheath 26 may be a laser cut stainless steel tube with a polymer jacket (e.g., an inner and/or outer layer of polymer) configured to provide strength, pushability, and/or compression resistance.

The proximal end region 58 of the inner sheath 26 may be attached to a flared proximal gripping member 62. The proximal gripping member 62 may be disposed exterior of the handle 14 proximal to a proximal end 16 of the handle 14. In some embodiments, the flared shape of the proximal gripping member 62 and other structural features may allow the proximal gripping member 62 to function as a guidewire introducer or funnel that may assist a clinician in placing, holding, removing, and/or exchanging a guidewire extending through the inner sheath 26. However, this is not required. It is further contemplated that the flared shape of the proximal gripping member 62 may also function as a mechanical stop to limit distal movement of the inner sheath 26. However, it is contemplated that the proximal gripping member 62 need not have a flared shape. In some cases, the proximal gripping member 62 may have a substantially uniform outer diameter. In other embodiments, the proximal gripping member 62 may be omitted.

The elongate shaft 12 may further include an inner shaft 32 extending from a proximal end 33 to a distal end region 34. The proximal end 33 may extend proximally through the handle 14 and may be coupled to an enlarged handgrip 64. However, it is contemplated that the handgrip 64 need not have an enlarged shape relative to the inner shaft 32. It is contemplated that the handgrip 64 may take any shape desired. In some embodiments, the handgrip 64 may be sized and shaped to allow the handgrip 64 to function as a guidewire introducer or funnel that may assist a clinician in placing, holding, removing, and/or exchanging a guidewire extending through the inner sheath 26. In other embodiments, the handgrip 64 may be omitted. While not explicitly shown, in some cases, the proximal end 33 of the inner shaft 32 may be coupled to a hypotube. The hypotube may then be actuated to distally advance or proximally retract the inner shaft 32, as desired.

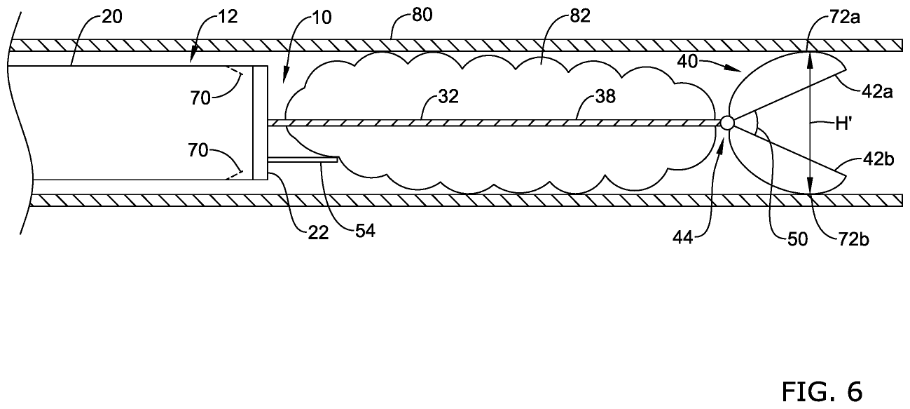

The inner shaft 32 may be positioned radially inwards of the inner sheath 26 and slidably disposed within a lumen 36 thereof. For example, the inner shaft 32 may be configured to be axially and/or rotationally displaced relative to the inner sheath 26 and/or the outer catheter 20. The inner shaft 32 may be releasably coupled to the inner sheath 26. For example, when the inner shaft 32 and the inner sheath 26 are coupled, the inner shaft 32 and the inner sheath 26 may be moved together and when the inner shaft 32 and the inner sheath 26 are uncoupled, the inner shaft 32 and the inner sheath 26 may be moved independently of one another (e.g., via the handgrip 64 or via the proximal gripping member 62, respectively). The inner shaft 32 may be formed from a core wire having a helically wound coil 38 (see, for example, FIG. 6) disposed about an outer surface of the core wire for optimal compression resistance when advanced through a clot.

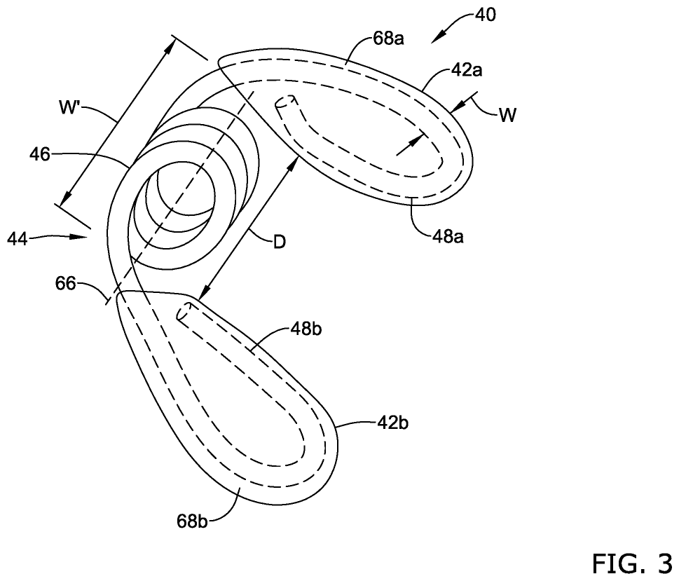

A deployable anchor mechanism 40 may extend distally from the distal end region 34 of the inner shaft 32. FIG. 3 illustrates a perspective view of the illustrative anchor mechanism 40. The anchor mechanism 40 may include a first portion or wing 42a and a second portion or wing 42b (collectively, 42). The wings 42 may be actuatable between an open configuration (see, for example, FIG. 3 and FIG. 6) and a closed configuration, as shown in FIG. 2. A torsion spring 44 may be configured to bias the wings 42 into the open configuration while the inner sheath 26 may bias the wings 42 into the closed configuration. It is contemplated that other types of springs or actuation mechanisms may be used in place of or in addition to the torsion spring 44. The anchor mechanism 40 may be formed from a variety of materials including, but not limited to, polymers, metals, metal alloys, etc. In some embodiments, the anchor mechanism 40 may be formed from a shape memory material, such as, but not limited to, shape memory alloys or polymers or any other self-expandable materials. When employing such shape-memory materials, the anchor mechanism 40 may be heat set in the expanded state and then compressed to fit within the inner sheath 26, for example. Alternatively, external forces such as, but not limited to, pneumatic methods, compressed fluid, pull wires, push wires, or the like may also be employed to expand the anchor mechanism 40.

The torsion spring 44 may be formed from a helically wound wire having a helically wound portion 46 and one or more free ends 48a, 48b (collectively, 48) extending radially from the helically wound portion 46. While the helically would portion 46 is illustrated as including approximately 3 loops, the helically wound portion 46 may include any number of loops, including less than one complete loop, one loop, two loop, three loops, four or more loops, etc. It is contemplated that the number of loops in the helically wound portion 46 may be varied to vary a distance D between the wings 42. In the illustrated embodiment, the free ends 48 turn back on themselves to form a partial oblong loop. It is contemplated that the free ends 48 may take other shapes, as desired. For example, the free ends 48 may take the shape of a curve which does not fold back on itself. In other examples, the free ends 48 may be bent or curved to form other cross-sectional shapes, including but not limited to, circular, triangular, square, rectangular, polygonal, eccentric, etc.

In an unbiased configuration, the free ends 48 may extend at a first angle 50 (see, for example, FIG. 6) relative to one another. The free ends 48 may be compressed or collapsed through rotations thereof. For example, the free ends 48 may be rotated radially inwards about a central axis 66 of the helically wound portion 46 to collapse the anchor mechanism 40 such that the free ends extend at a second angle 52 (see, for example, FIG. 2) relative to one another that is less than the first angle 50. When the free ends 48 are rotated from their original or unbiased configuration, the helically wound portion 46 may bias or push the free ends back to their original position when the anchor mechanism 40 is unconstrained. An externally applied force may maintain the free ends 48 in a biased configuration. For example, the inner sheath 26 may maintain the free ends 48 in a biased or collapsed configuration. As will be described in more detail herein, proximal retraction of the inner sheath 26 may allow the free ends 48 to assume their original, unbiased, configuration or to move towards the unbiased configuration. As the free ends 48 move, the wings 42 may move radially outward or pivot about the helically wound portion 46 in a manner similar to a jaw.

It is contemplated that the wings 42 may be formed from a portion of the torsion spring 44 (e.g., the free ends 48), as shown in FIG. 3, or the wings 42 may be a separate structure coupled to the free ends 48 of the torsion spring 44, as desired. In one example, the wings 42 may include a polymer jacket 68a, 68b (collectively, 68) formed over the free ends 48 of the torsion spring 44. In some examples, the polymer jacket 68 may extend between portions of the free end 48 and cover the opening of the loop to form a substantially solid wing. In other examples, the polymer jacket 68 may be a tubular coating disposed over an outer surface of the free ends 48 (e.g., a polymer coated wire) such that the opening in the loop remains free from the polymer coating. The surface of the wing 42 configured to be in contact with the vessel wall may be configured to not penetrate or puncture the vessel wall. It is further contemplated that an outer surface of the wings 42 may include serrations or texturing configured to grip an inner surface of a vessel when the wings 42 are opened or in an unbiased configuration. While the anchor mechanism 40 is illustrated as including two wings 42, the anchor mechanism 40 may include any number of wings 42 desired, including one, two, three, four, five, ten, or more. In some cases, the wings 42 may at least partially overlap in the collapsed and/or expanded configurations. For example, the wings 42 may take the form of a plurality of interconnected overlapping wings 42 coupled to a central base and fold or pivot relative to the base to move between a radially collapsed and a radially expanded configuration. In an expanded configuration, the outer diameter of the overlapping wings may conform to the interior of a vessel.

It is further contemplated that while a collective height II of the wings 42 (see, for example, FIG. 2) in the collapsed configuration may be similar to an inner diameter of the inner sheath 26, a width W of the individual wings 42 (see, for example, FIG. 3) and/or a width W′ of the anchor mechanism 40 may be less than the inner diameter of the inner sheath 26. This may allow other devices to pass distally beyond the anchor mechanism 40. For example, the mechanical thrombectomy system 10 may further include a guidewire 54 disposed within the lumen 36 of the inner sheath 26. The guidewire 54 may extend from a proximal end 55 configured to remain outside the body to a distal end 56 configured to be advanced distally beyond a distal end 28 of the inner sheath 26 and/or a distal end 22 of the outer catheter 20. In other embodiments, the guidewire 54 may be disposed within the lumen 30 of the outer catheter 20. It is contemplated that the guidewire 54 may be laterally offset from a central longitudinal axis of the elongate shaft 12, although this is not required. The guidewire 54 may be disposed within the lumen 36 of the inner sheath 26 or the lumen 30 of the outer catheter 20 along essentially an entire length the entire length of the elongate shaft 12 such that mechanical thrombectomy system 10 resembles traditional “over-the-wire” catheters. Alternatively, guidewire 54 may be disposed within the lumen 36 of the inner sheath 26 or the lumen 30 of the outer catheter 20 along only a portion of the elongate shaft 12 such that the mechanical thrombectomy system 10 resembles “single-operator-exchange” or “rapid-exchange” catheters. The elongate shaft 12 may be advanced over the guidewire 54 to the desired target location in the vasculature.

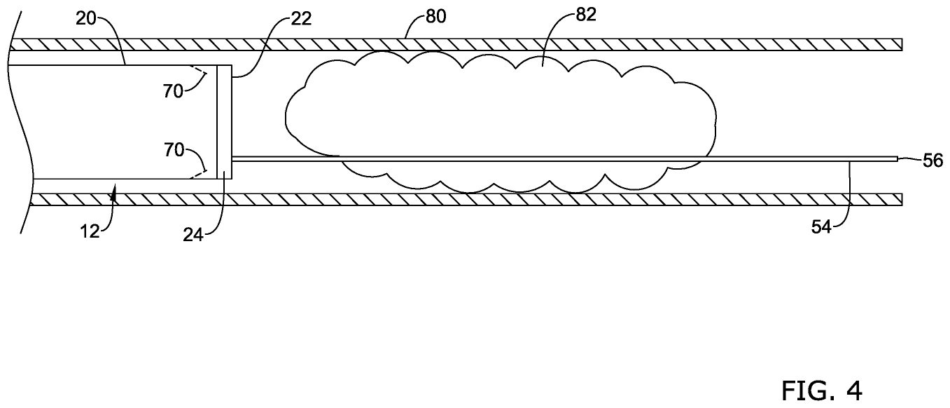

An illustrative method for using the mechanical thrombectomy system 10 to remove a clot or other debris 82 from within a vessel 80 will be described with respect to FIGS. 4-8. Referring first to FIG. 4, the guidewire 54 may be advanced through the vasculature to the target location and then the elongate shaft 12 advanced over the guidewire 54, although this is not required. In some embodiments, the guidewire 54 and the elongate shaft 12 may be tracked together, with the guidewire 54 leading the elongate shaft 12 (e.g., advance the guidewire 54 a distance, then advance the elongate shaft 12 over the guidewire 54 approximately the same distance. The guidewire 54 may be distally advanced through the clot 82 so that the distal end 56 of the guidewire 54 is distal to the clot 82, as shown in FIG. 4. The outer catheter 20, the inner sheath 26, and the inner shaft 32 may be advanced simultaneously over the guidewire 54 until the distal end 22 of the outer catheter 20 is adjacent to and proximal to the clot 82. In other embodiments, the outer catheter 20, the inner sheath 26, and the inner shaft 32 may be advanced individually or in other arrangements, as desired.

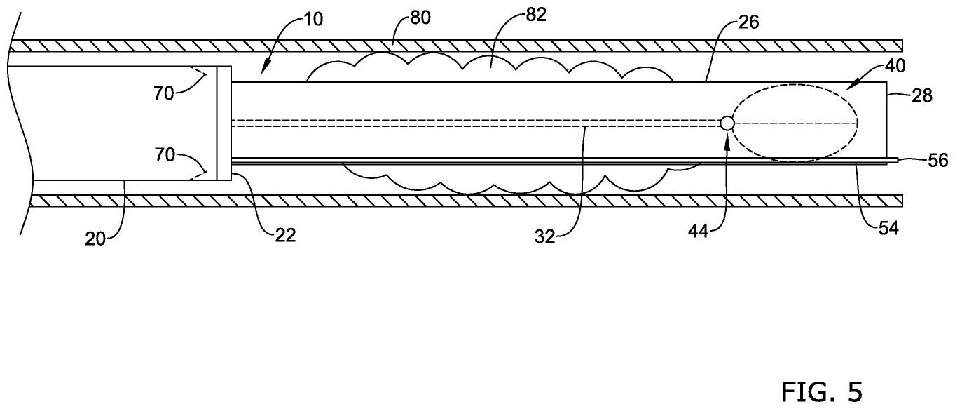

Referring now to FIG. 5, once the distal end 22 of the outer catheter 20 is adjacent to and proximal to the clot 82, the inner sheath 26 may be distally advanced through the clot 82 such that the distal end 28 of the inner sheath 26 is distal to the clot 82. The inner shaft 32 and the anchor mechanism 40 may be advanced with the inner sheath 26 or subsequent to the advancement of the inner sheath 26 until the inner shaft 32 and the anchor mechanism 40 are distal to the clot 82. Next, the inner sheath 26 may be proximally retracted, as shown in FIG. 6. In some cases, the inner sheath 26 may be proximally retracted through actuation of the proximal gripping member 62, or other actuation mechanism, while the inner shaft 32 is held stationary. It is contemplated that in some cases, the inner sheath 26 may be held stationary while the inner shaft 32 is distally advanced distally beyond the distal end 28 of the inner sheath 26. As the inner sheath 26 is proximally retracted (or the inner shaft 32 distally advanced), the radial compressive force of the inner sheath 26 on the anchor mechanism 40 may be released. In the absence of the compressive force, the anchor mechanism 40 may transition into the deployed configuration. For example, the free ends 48 of the torsion spring 44 may rotate radially outward. In the deployed configuration, the wings 42 may have a collective height H′ that is greater than the height H of the collapsed configuration and approximately the same as the inner diameter of the vessel 80. In some cases, the inner wall of the vessel 80 may preclude or prevent the anchor mechanism 40 from returning to a fully expanded configuration. Thus, the deployed configuration may be any configuration between the collapsed delivery configuration and a fully expanded configuration (e.g., the configuration of the anchor mechanism 40 in the absence of any applied force). In the deployed configuration, the outer surfaces 72a, 72b (collectively, 72) of the wings 42 may engage the inner surface of the vessel wall 80. As described above, the wings 42 may not puncture the vessel wall 80. Rather the wings 42 may frictionally engage the vessel 80 to anchor the mechanical thrombectomy system 10 to hold the inner shaft 32 in a fixed position. It is contemplated that the anchor mechanism 40 may provide leverage to help the outer catheter 20 pass through the clot 82.

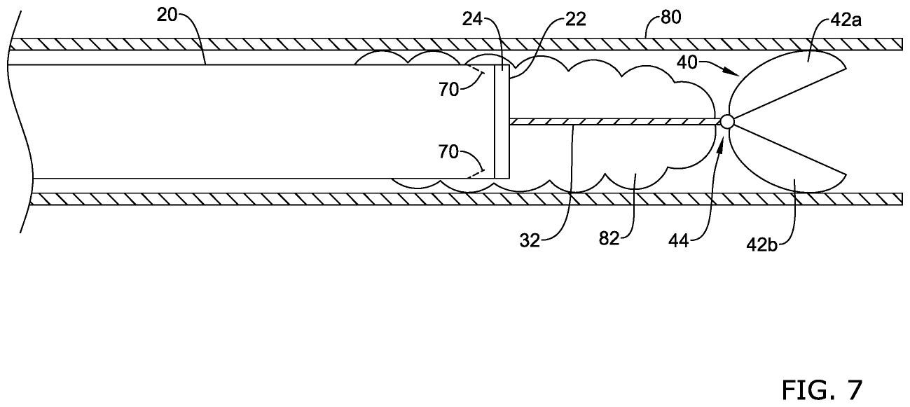

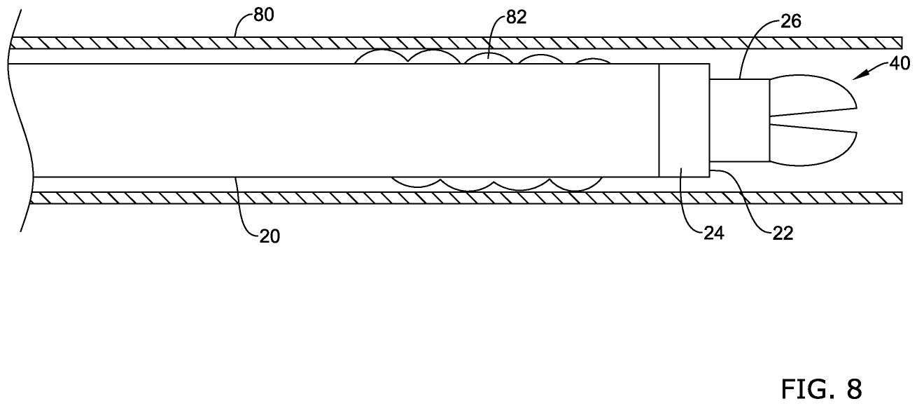

Referring now to FIG. 7, once the anchor mechanism 40 is deployed, the outer catheter 20 may be distally advanced through the clot 82. As the outer catheter 20 is distally advanced the distal end 22, the radiopaque marker 24 and/or the cutting member 70 cut through the clot 82 and the clot 82 is drawn into the lumen 30 of the outer catheter 20. In some cases, aspiration may be used to help draw the clot into the lumen 30. Once the distal end 22 of the outer catheter 20 is distal to the distal end of the clot 82, the inner sheath 26 may be distally advanced to collapse and re-sheath the anchor mechanism 40, as shown in FIG. 8. In some embodiments, the outer catheter 20 may be distally advanced to collapse and re-sheath the anchor mechanism 40. In some cases, the outer catheter 20 may be proximally retracted and repositioned within the vessel 80 to capture more of the clot 82 without relocation of the anchor mechanism 40. For example, the outer catheter 20 may be repositioned about a circumference of the vessel 80 to fully capture the clot 82. It is contemplated that the outer catheter 20 may be proximally retracted, repositioned, and distally advanced as many times as needed or as desired to capture the clot. It is contemplated that the number of times the outer catheter 20 needs to be repositioned to fully capture the clot 82 may depend on diameter of the vessel 80 and/or the diameter of the outer catheter 20. The mechanical thrombectomy system 10 may then be removed from the body or advanced to another target location to remove another clot, as desired.

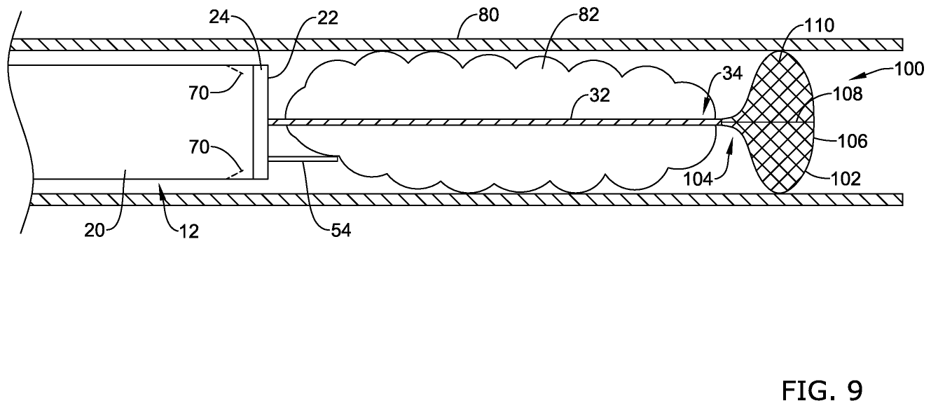

FIG. 9 is a schematic side view of the illustrative mechanical thrombectomy system 10 in a partially deployed configuration and having an alternative anchor mechanism 100. The anchor mechanism 100 may include an expandable basket 102 coupled to the distal end region 34 of the inner shaft 32. The expandable basket 102 may be configured to transition between a collapsed configuration and an expanded configuration (FIG. 9). A proximal end 104 of the expandable basket 102 may be coupled to the inner shaft 32 while a distal end 106 of the expandable basket 102 may be coupled to a central wire 108. The central wire 108 may extend proximally to a proximal end configured to remain outside the body.

The expandable basket 102 may have a woven structure, fabricated from a number of filaments or struts 110 forming a generally tubular wall that may be deformed to radially expand, as shown in FIG. 9. In some embodiments, the expandable basket 102 may be knitted or braided with a single filament or strut interwoven with itself and defining open cells extending through the thickness of the tubular wall of the expandable basket 102. In other embodiments, the expandable basket 102 may be braided with several filaments or struts interwoven together and defining open cells extending along a length and around the circumference of the tubular wall of the expandable basket 102. In another embodiment, the expandable basket 102 may be knitted. In yet another embodiment, the expandable basket 102 may be of a knotted type. In still another embodiment, the expandable basket 102 may be a laser cut tubular member. A laser cut tubular member may have an open and/or closed cell geometry including one or more interconnected monolithic filaments or struts defining open cells therebetween, with the open cells extending along a length and around the circumference of the tubular wall.

The expandable basket 102 may be self-expandable or may require external force to expand from a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the expandable basket 102 may be heat set in the expanded state and then compressed to fit within the inner sheath 26, for example. In another embodiment, a spring may be provided to effect expansion. Alternatively, external forces such as, but not limited to, pneumatic methods, compressed fluid, pull wires, push wires, or the like may also be employed to expand the expandable basket 102. It is contemplated that nickel-titanium alloys may enable kink-resistant folding and self-expansion. In other examples, magnetic alloys, metals, metal alloys, polymers, composites, etc. may be used to form the expandable basket 102.

In other instances, a manual force applied to the inner shaft 32 may manipulate or actuate the expandable basket 102 between the expanded and collapsed state. For example, an actuation element may include a central wire 108 that extends through the inner shaft 32 and the expandable basket 102 and is coupled to a distal end 106 of the expandable basket 102. According to this embodiment, a pulling force exerted proximally on the wire 108 may allow the expandable basket 102 to expand and move the expandable basket 102 into an expanded state. A pushing force exerted distally on the wire 108 may elongate the expandable basket 102 and/or otherwise shift the expandable basket 102 to a compressed or elongated state. Other actuation mechanisms may also be utilized. For example, in some cases, the inner shaft 32 may be actuated relative to the central wire 108.

The anchor mechanism 100 may anchor the mechanical thrombectomy system 10 to allow for removal of a clot 82 in a similar manner to that described with respect to FIGS. 4-8. For example, the guidewire 54 may be advanced through the vasculature to the target location and then the elongate shaft 12 advanced over the guidewire 54, although this is not required. In some embodiments, the guidewire 54 and the elongate shaft 12 may be tracked together, with the guidewire 54 leading the elongate shaft 12 (e.g., advance the guidewire 54 a distance, then advance the elongate shaft 12 over the guidewire 54 approximately the same distance. The guidewire 54 may be distally advanced through the clot 82 so that the distal end 56 of the guidewire 54 is distal to the clot 82. The outer catheter 20, the inner sheath 26, and the inner shaft 32 may be advanced simultaneously over the guidewire 54 until the distal end 22 of the outer catheter 20 is adjacent to and proximal to the clot 82. In other embodiments, the outer catheter 20, the inner sheath 26, and the inner shaft 32 may be advanced individually or in other arrangements, as desired.

Once the distal end 22 of the outer catheter 20 is adjacent to and proximal to the clot 82, the inner sheath 26 may be distally advanced through the clot 82 such that the distal end 28 of the inner sheath 26 is distal to the clot 82. The inner shaft 32 and the anchor mechanism 100 may be advanced with the inner sheath 26 or subsequent to the advancement of the inner sheath 26 until the inner shaft 32 and the anchor mechanism 100 are distal to the clot 82. Next, the inner sheath 26 may be proximally retracted. In some cases, the inner sheath 26 may be proximally retracted through actuation of the proximal gripping member 62, or other actuation mechanism, while the inner shaft 32 is held stationary. It is contemplated that in some cases, the inner sheath 26 may be held stationary while the inner shaft 32 is distally advanced distally beyond the distal end 28 of the inner sheath 26. As the inner sheath 26 is proximally retracted (or the inner shaft 32 distally advanced), the radial compressive force of the inner sheath 26 on the anchor mechanism 100 may be released. In the absence of the compressive force, the anchor mechanism 100 may transition without user intervention into the deployed configuration. In other embodiments, the user may proximally retract the central wire 108 to expand the basket 102. The expandable basket 102 may expand to have an outer dimension that is approximately the same as the inner diameter of the vessel 80. In some cases, the inner wall of the vessel 80 may preclude or prevent the anchor mechanism 100 from returning to a fully expanded configuration. Thus, the deployed configuration may be any configuration between the collapsed delivery configuration and a fully expanded configuration. In the deployed configuration, an outer perimeter of the expandable basket 102 may engage the inner surface of the vessel wall 80. The expandable basket 102 may not puncture the vessel wall 80. Rather the expandable basket 102 may frictionally engage the vessel 80 to anchor the mechanical thrombectomy system 10 to hold the inner shaft 32 in a fixed position. It is contemplated that the anchor mechanism 100 may provide leverage to help the outer catheter 20 pass through the clot 82.

Once the anchor mechanism 100 is deployed, the outer catheter 20 may be distally advanced through the clot 82. As the outer catheter 20 is distally advanced the distal end 22, the radiopaque marker 24 and/or the cutting member 70 cut through the clot 82 and the clot 82 is drawn into the lumen 30 of the outer catheter 20. In some cases, aspiration may be used to help draw the clot into the lumen 30. Once the distal end 22 of the outer catheter 20 is distal to the distal end of the clot 82, the inner sheath 26 may be distally advanced to collapse and re-sheath the anchor mechanism 100. Alternatively, or additionally, the central wire 108 may be distally advanced to collapse the anchor mechanism 100. In some embodiments, the outer catheter 20 may be distally advanced to collapse and re-sheath the anchor mechanism 100. In some cases, the outer catheter 20 may be proximally retracted and repositioned within the vessel 80 to capture more of the clot 82 without relocation of the anchor mechanism 100. For example, the outer catheter 20 may be repositioned about a circumference of the vessel 80 to fully capture the clot 82. It is contemplated that the outer catheter 20 may be proximally retracted, repositioned, and distally advanced as many times as needed or as desired to capture the clot. It is contemplated that the number of times the outer catheter 20 needs to be repositioned to fully capture the clot 82 may depend on diameter of the vessel 80 and/or the diameter of the outer catheter 20. The mechanical thrombectomy system 10 may then be removed from the body or advanced to another target location to remove another clot, as desired.

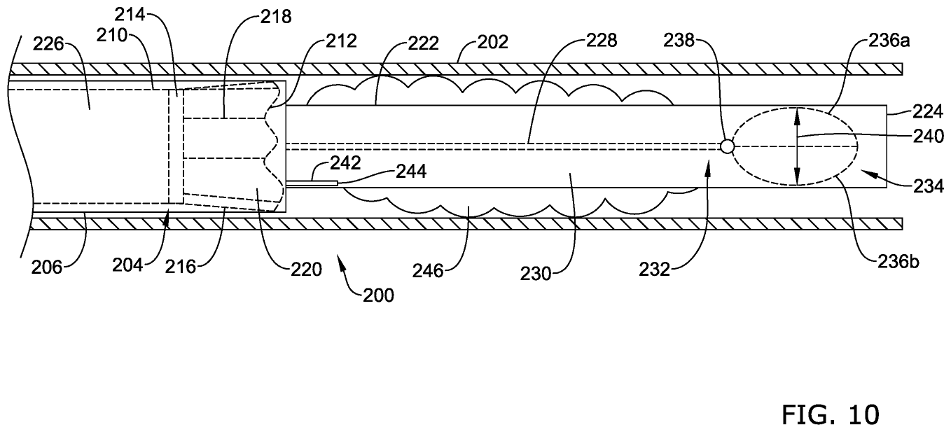

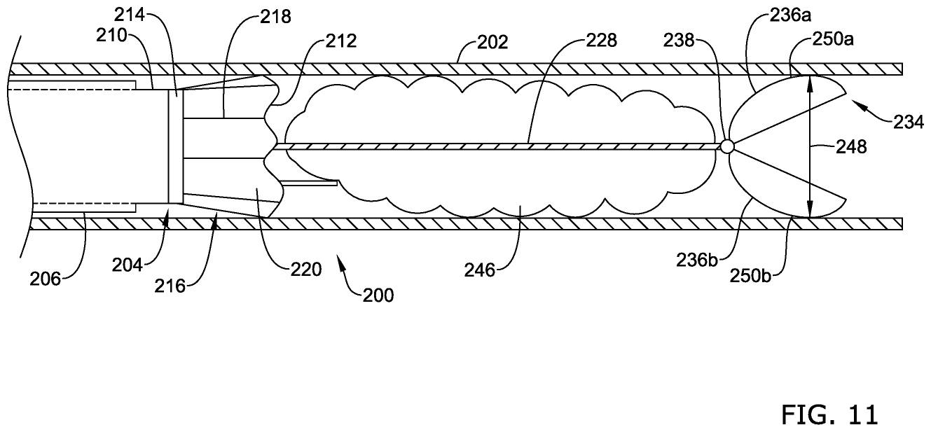

FIGS. 10 and 11 depict a schematic side view of another illustrative clot entrapment system or mechanical thrombectomy system 200 in a partially deployed configuration within a vessel 202. The mechanical thrombectomy system 200 may include an elongate shaft 204. While not explicitly shown, a handle similar in form and function to the handle 14 described herein may be coupled to a proximal end region of the elongate shaft 204. In general, the mechanical thrombectomy system 200 may be used to entrap any type of clot, including newer, acute thrombotic material along with older, sub-acute and chronic material, or the like, within a body lumen of a patient. The body lumen may be a blood vessel located near the heart (e.g., within or near a cardiac vessel), within a peripheral vessel, within a neurological vessel, or at any other suitable location. In some cases, the mechanical thrombectomy system 200 may include one or more distal anchors configured to anchor a distal end region of the mechanical thrombectomy system 200 while the clot is being removed.

The elongate shaft 204 may include a number of elongate structures that are co-axially arranged (e.g., sharing a common longitudinal axis) or that are at least partially disposed one inside of another and not necessarily sharing a common longitudinal axis. In some cases, one or more of the elongate structures may be radially offset from a central longitudinal axis of the mechanical thrombectomy system 200 The elongate shaft 204 may include an outer sheath 206 coupled to the handle. The outer sheath 206 may extend distally from a proximal end coupled to the handle to a distal end 208 configured to be advanced within a body lumen. In some embodiments, the outer sheath 206 may be a tubular polymer, may be made up of a coil or braid reinforced polymer, or may be a polymer encapsulated laser cut metallic tube. For example, the outer sheath 206 may be a laser cut stainless steel tube with a polymer jacket (e.g., an inner and/or outer layer of polymer) configured to provide strength, pushability, and/or compression resistance. The outer sheath 206 may be provided with or without a lubricious liner, such as, but not limited to, polytetrafluoroethylene (PTFE) or perfluoroalkoxy alkane (PFA). An exterior of the outer sheath 206 may include a lubricious additive. Polymers for the outer sheath 206 may include, but are not limited to, PEBAX®, urethane, polyimide, polyamide, a combination thereof, or the like.

The elongate shaft 204 may also include an outer catheter or intermediate sheath 210 movably coupled to the handle. The intermediate sheath 210 may extend distally from a proximal end disposed within the handle to a distal end 212 configured to be advanced within a body lumen. The intermediate sheath 210 may be disposed radially inwards of the outer sheath 206. In some embodiments, the intermediate sheath 210 may be a tubular polymer, may be made up of a coil or braid reinforced polymer, or may be a polymer encapsulated laser cut metallic tube. For example, the intermediate sheath 210 may be a laser cut stainless steel tube with a polymer jacket (e.g., an inner and/or outer layer of polymer) configured to provide strength, pushability, and/or compression resistance. In some embodiments, a radiopaque marker band 214 may be positioned at or adjacent to the distal end 212. The marker band 214 may allow the distal end 212 of the intermediate sheath 210 to be fluoroscopically visualized during advancement of the mechanical thrombectomy system 200. It is contemplated that the marker band 214 may increase the pushability of the intermediate sheath 210 through a lesion such as, but not limited to, acute thrombotic material, sub-acute thrombotic material, and chronic material. The intermediate sheath 210 may be provided with or without a lubricious liner, such as, but not limited to, polytetrafluoroethylene (PTFE) or perfluoroalkoxy alkane (PFA). An exterior of the intermediate sheath 210 may include a lubricious additive. Polymers for the intermediate sheath 210 may include, but are not limited to, PEBAX®, urethane, polyimide, polyamide, a combination thereof, or the like.

The intermediate sheath 210 may further include an expandable distal end region 216. The expandable distal end region 216 may include a plurality of support members 218 with a membrane 220 extending between and coupled to the support members 218. At least some of the support members 218 may extending in a generally longitudinal direction. In some examples, one or more of the support members 218 may extend about a circumference or a portion of the circumference of the intermediate sheath 210. The support members 218 may be movable between a collapsed configuration (FIG. 10) and an expanded configuration (FIG. 11). For example, the support members 218 may be self-expanding. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the support members may be heat set in the expanded state and then compressed to fit within the outer sheath 206, for example. It is contemplated that the expandable distal end region 216 may take other shapes and/or configurations, as desired.

The proximal end region of the intermediate sheath 210 may be attached to a gear rack assembly disposed within the handle with a fastener or clip (not explicitly shown). As the intermediate sheath 210 is coupled to the gear rack assembly, proximal movement of the gear rack assembly may result in analogous proximal movement of the intermediate sheath 210 and distal movement of the gear rack assembly may result in analogous distal movement of the intermediate sheath 210. The gear rack assembly may include a plurality of teeth or gears (not explicitly shown). In practice, the teeth may be configured to engage with corresponding teeth or gears on a thumbwheel of the handle. Consequently, rotation of the thumbwheel, via gearing thereof with the gears of the gear rack assembly, can be utilized to proximally retract and/or distally advance the gear rack assembly and, thus, the intermediate sheath 210. Other structural arrangements may be utilized to accomplish proximal retraction and/or distal advancement of the gear rack assembly through the actuation of the thumbwheel or any other suitable actuation member.

While not explicitly shown, the intermediate sheath 210 may optionally include one or more cutting members. The cutting member may extend radially inward from an inner surface of the intermediate sheath 210. In some cases, the cutting member may be a circular blade extending about an entire inner circumference of the intermediate sheath 210. In other embodiments, the cutting member may extend about less than entire circumference of the intermediate sheath 210. In yet other embodiments, the cutting member may include two or more cutting members uniformly or eccentrically positioned about an inner surface of the intermediate sheath 210. When so provided, the cutting member may help cut through the clot. The cutting member may be positioned proximal to the distal end 212 of the intermediate sheath 210. This may help prevent or reduce damage to the blood vessel during advancement of the mechanical thrombectomy system 200. However, this is not required. In some cases, the cutting member may be positioned at or distal to the distal end 212.

The elongate shaft 204 may also include an inner sheath 222 extending from a proximal end region (not explicitly shown) configured to remain outside the body, through the handle to a distal end 224. The inner sheath 222 may be positioned radially inwards of the intermediate sheath 210 and slidably disposed within a lumen 226 thereof. For example, the inner sheath 222 may be configured to be axially and/or rotationally displaced relative to the intermediate sheath 210. In some embodiments, the inner sheath 222 may be a tubular polymer, may be made up of a coil or braid reinforced polymer, or may be a polymer encapsulated laser cut metallic tube. For example, the inner sheath 222 may be a laser cut stainless steel tube with a polymer jacket (e.g., an inner and/or outer layer of polymer) configured to provide strength, pushability, and/or compression resistance.

The proximal end region of the inner sheath 222 may be attached to a flared proximal gripping member (not explicitly shown). The proximal gripping member may be disposed exterior of the handle proximal to a proximal end of the handle. In some embodiments, the flared shape of the proximal gripping member and other structural features may allow the proximal gripping member to function as a guidewire introducer or funnel that may assist a clinician in placing, holding, removing, and/or exchanging a guidewire extending through the inner sheath 222. It is further contemplated that the flared shape of the proximal gripping member may also function as a mechanical stop to limit distal movement of the inner sheath 222. However, it is contemplated that the proximal gripping member need not have a flared shape. In some cases, the proximal gripping member may have a substantially uniform outer diameter. In other embodiments, the proximal gripping member may be omitted.

The elongate shaft 204 may further include an inner shaft 228 extending from a proximal end (not explicitly shown) to a distal end region 232. The proximal end may extend proximally through the handle and may be coupled to an enlarged handgrip (not explicitly shown). However, it is contemplated that the handgrip need not have an enlarged shape relative to the inner shaft 228. It is contemplated that the handgrip may take any shape desired. In other embodiments, the handgrip may be omitted. While not explicitly shown, in some cases, the proximal end of the inner shaft 228 may be coupled to a hypotube. The hypotube may then be actuated to distally advance or proximally retract the inner shaft 228, as desired.

The inner shaft 228 may be positioned radially inwards of the inner sheath 222 and slidably disposed within a lumen 230 thereof. For example, the inner shaft 228 may be configured to be axially and/or rotationally displaced relative to the inner sheath 222 and/or the intermediate sheath 210. The inner shaft 228 may be releasably coupled to the inner sheath 222. For example, when the inner shaft 228 and the inner sheath 222 are coupled, the inner shaft 228 and the inner sheath 222 may be moved together and when the inner shaft 228 and the inner sheath 222 are uncoupled, the inner shaft 228 and the inner sheath 222 may be moved independently of one another (e.g., via the handgrip or via the proximal gripping member, respectively). The inner shaft 228 may be formed from a core wire having a helically wound coil (not explicitly shown) disposed about an outer surface of the core wire for optimal compression resistance when advanced through a clot.

A deployable anchor mechanism 234 may extend distally from the distal end region 232 of the inner shaft 228. The illustrated anchor mechanism 234 may be similar in form and function to the anchor mechanism 40 described herein. Alternatively, the anchor mechanism 234 may be similar in form and function to the anchor mechanism 100 described herein. The anchor mechanism 234 may include a first portion or wing 236a and a second portion or wing 236b (collectively, 236). The wings 236 may be actuatable between an open configuration (see, for example, FIG. 11) and a closed configuration (see, for example, FIG. 10). A torsion spring 238 may be configured to bias the wings 236 into the open configuration while the inner sheath 222 may bias the wings 236 into the closed configuration. It is contemplated that other types of springs or actuation mechanisms may be used in place of or in addition to the torsion spring 238. The anchor mechanism 234 may be formed from a variety of materials including, but not limited to, polymers, metals, metal alloys, etc. In some embodiments, the anchor mechanism 234 may be formed from a shape memory material, such as, but not limited to, shape memory alloys or polymers or any other self-expandable materials. When employing such shape-memory materials, the anchor mechanism 234 may be heat set in the expanded state and then compressed to fit within the inner sheath 222, for example. Alternatively, external forces such as, but not limited to, pneumatic methods, compressed fluid, pull wires, push wires, or the like may also be employed to expand the anchor mechanism 234.

The torsion spring 238 may be formed from a helically wound wire having a helically wound portion and free ends extending radially from the helically wound portion, as described above with respect to anchor mechanism 40. In some embodiments, the free ends may turn back on themselves to form a partial oblong loop. However, the free ends may take other shapes, as desired. For example, the free ends may take the shape of a curve which does not fold back on itself. In other examples, the free ends may be bent or curved to form other cross-sectional shapes, including but not limited to, circular, triangular, square, rectangular, polygonal, eccentric, etc.

In an unbiased configuration, the free ends may extend at a first angle relative to one another. The free ends may be compressed or collapsed through rotations thereof. For example, the free ends may be rotated radially inwards about a central axis of the helically wound portion to collapse the anchor mechanism 234 such that the free ends extend at a second angle relative to one another that is less than the first angle. When the free ends are rotated from their original or unbiased configuration, the helically wound portion may bias or push the free ends back to their original position when the anchor mechanism 234 is unconstrained. An externally applied force may maintain the free ends in a biased configuration. For example, the inner sheath 222 may maintain the free ends in a biased or collapsed configuration. As will be described in more detail herein, proximal retraction of the inner sheath 222 may allow the free ends to assume their original, unbiased, configuration or to move towards the unbiased configuration. As the free ends move, the wings 236 may move radially outward or pivot about the helically wound portion in a manner similar to a jaw.

It is contemplated that the wings 236 may be formed from a portion of the torsion spring 238 (e.g., the free ends) or the wings 236 may be a separate structure coupled to the free ends of the torsion spring 238, as desired. In one example, the wings may include a polymer jacket formed over the free ends of the torsion spring. In some examples, the polymer jacket may extend between portions of the free end and cover the opening of the loop to form a substantially solid wing. In other examples, the polymer jacket may be a tubular coating disposed over an outer surface of the free ends (e.g., a polymer coated wire) such that the opening in the loop remains free from the polymer coating. The surface of the wing 236 configured to be in contact with the vessel wall may be configured to not penetrate or puncture the vessel wall. It is further contemplated that an outer surface of the wings 236 may include serrations or texturing configured to grip an inner surface of a vessel when the wings 236 are opened or in an unbiased configuration. While the anchor mechanism 234 is illustrated as including two wings 236, the anchor mechanism 234 may include any number of wings 236 desired, including one, two, three, four, five, ten, or more. In some cases, the wings 236 may at least partially overlap in the collapsed and/or expanded configurations. For example, the wings 236 may take the form of a plurality of interconnected overlapping wings 236 coupled to a central base and fold or pivot relative to the base to move between a radially collapsed and a radially expanded configuration. In an expanded configuration, the outer diameter of the overlapping wings may conform to the interior of a vessel.

Itis further contemplated that while a collective height 240 of the wings 236 (see, for example, FIG. 10) in the collapsed configuration may be similar to an inner diameter of the inner sheath 222, a width of the individual wings 236 and/or a width of the anchor mechanism 234 may be less than the inner diameter of the inner sheath 222. This may allow other devices to pass distally beyond the anchor mechanism 234. For example, the mechanical thrombectomy system 200 may further include a guidewire 242 disposed within the lumen 230 of the inner sheath 222. The guidewire 242 may extend from a proximal end configured to remain outside the body to a distal end 244 configured to be advanced distally beyond a distal end 224 of the inner sheath 222 and/or a distal end 212 of the intermediate sheath 210. In other embodiments, the guidewire 242 may be disposed within the lumen 226 of the intermediate sheath 210. It is contemplated that the guidewire 242 may be laterally offset from a central longitudinal axis of the elongate shaft 204, although this is not required. The guidewire 242 may be disposed within the lumen 230 of the inner sheath 222 or the lumen 226 of the intermediate sheath 210 along essentially an entire length the entire length of the elongate shaft 204 such that mechanical thrombectomy system 200 resembles traditional “over-the-wire” catheters. Alternatively, guidewire 242 may be disposed within the lumen 230 of the inner sheath 222 or the lumen 226 of the intermediate sheath 210 along only a portion of the elongate shaft 204 such that the mechanical thrombectomy system 200 resembles “single-operator-exchange” or “rapid-exchange” catheters. The elongate shaft 204 may be advanced over the guidewire 242 to the desired target location in the vasculature.

The anchor mechanism 234 may anchor the mechanical thrombectomy system 200 to allow for removal of a clot 246 in a similar manner to that described with respect to FIGS. 4-8. The guidewire 242 may be advanced through the vasculature to the target location and then the elongate shaft 204 advanced over the guidewire 242, although this is not required. In some embodiments, the guidewire 242 and the elongate shaft 204 may be tracked together, with the guidewire 242 leading the elongate shaft 204 (e.g., advance the guidewire 242 a distance, then advance the elongate shaft 204 over the guidewire 242 approximately the same distance. The guidewire 242 may be distally advanced through the clot 246 so that the distal end 244 of the guidewire 242 is distal to the clot 246. The outer sheath 206, the intermediate sheath 210, the inner sheath 222, and the inner shaft 228 may be advanced simultaneously over the guidewire 242 until the distal end 208 of the outer sheath 206 and the distal end 212 of the intermediate sheath 210 are adjacent to and proximal to the clot 246. In other embodiments, the outer sheath 206, the intermediate sheath 210, the inner sheath 222, and the inner shaft 228 may be advanced individually or in other arrangements, as desired.

Once the distal end 208 of the outer sheath 206 and the distal end 212 of the intermediate sheath 210 are adjacent to and proximal to the clot 246, the inner sheath 222 may be distally advanced through the clot 246 such that the distal end 224 of the inner sheath 222 is distal to the clot 246. The inner shaft 228 and the anchor mechanism 234 may be advanced with the inner sheath 222 or subsequent to the advancement of the inner sheath 222 until the inner shaft 228 and the anchor mechanism 234 are distal to the clot 246. Next, the inner sheath 222 may be proximally retracted. In some cases, the inner sheath 222 may be proximally retracted through actuation of the proximal gripping member, or other actuation mechanism, while the inner shaft 228 is held stationary. It is contemplated that in some cases, the inner sheath 222 may be held stationary while the inner shaft 228 is distally advanced distally beyond the distal end 224 of the inner sheath 222. As the inner sheath 222 is proximally retracted (or the inner shaft 228 distally advanced), the radial compressive force of the inner sheath 222 on the anchor mechanism 234 may be released. In the absence of the compressive force, the anchor mechanism 234 may transition into the deployed configuration, as shown in FIG. 11. For example, the free ends of the torsion spring 238 may rotate radially outward. In the deployed configuration, the wings 236 may have a collective height 248 that is greater than the height 240 of the collapsed configuration and approximately the same as the inner diameter of the vessel 202. In some cases, the inner wall of the vessel 202 may preclude or prevent the anchor mechanism 234 from returning to a fully expanded configuration. Thus, the deployed configuration may be any configuration between the collapsed delivery configuration and a fully expanded configuration (e.g., the configuration of the anchor mechanism 234 in the absence of any applied force). In the deployed configuration, the outer surfaces 250a, 250b (collectively, 250) of the wings 236 may engage the inner surface of the vessel wall 202. As described above, the wings 236 may not puncture the vessel wall 202. Rather the wings 236 may frictionally engage the vessel 202 to anchor the mechanical thrombectomy system 200 to hold the inner shaft 228 in a fixed position. It is contemplated that the anchor mechanism 234 may provide leverage to help the intermediate sheath 210 pass through the clot 246.

Once the anchor mechanism 234 is deployed, the outer sheath 206 may be proximally retracted to allow the expandable distal end region 216 to radially expand, as shown in FIG. 11. For example, once the radial compressive force of the outer sheath 206 on the support members 218 is released, the support members 218 may move towards their unbiased expanded configuration. The support members 218 may move radially outwards until an outer surface of the expandable distal end region 216 is in contact with an inner surface of the vessel wall 202. In some cases, the expandable distal end region 216 may contact the inner surface of the vessel wall 202 before the support members 218 return to an original unbiased configuration. The intermediate sheath 210 may then be distally advanced through the clot 246. As the intermediate sheath 210 is distally advanced the distal end 212 and/or a cutting member may cut through the clot 246 and the clot 246 is drawn into the lumen 226 of the intermediate sheath 210. In some cases, aspiration may be used to help draw the clot into the lumen 226. Once the distal end 212 of the intermediate sheath 210 is distal to the distal end of the clot 246, the inner sheath 222 may be distally advanced to collapse and re-sheath the anchor mechanism 234, as shown in FIG. 8. In some embodiments, the intermediate sheath 210 may be distally advanced to collapse and re-sheath the anchor mechanism 234. It is contemplated that the expandable distal end region 216 may allow for more of the clot 246 to be captured in a single pass than a system without an expandable distal end region 216. However, in some cases, the intermediate sheath 210 may be proximally retracted and repositioned within the vessel 202 to capture more of the clot 246 without relocation of the anchor mechanism 234. For example, the intermediate sheath 210 may be repositioned about a circumference of the vessel 202 to fully capture the clot 246. It is contemplated that the intermediate sheath 210 may be proximally retracted, repositioned, and distally advanced as many times as needed or as desired to capture the clot. It is contemplated that the number of times the intermediate sheath 210 needs to be repositioned to fully capture the clot 246 may depend on diameter of the vessel 202 and/or the diameter of the intermediate sheath 210. The mechanical thrombectomy system 200 may then be removed from the body or advanced to another target location to remove another clot, as desired.

The materials that can be used for the various components of the mechanical thrombectomy systems 10, 200 (and/or other systems disclosed herein) may include those commonly associated with medical devices. For simplicity purposes, the following discussion makes reference to the various components of the mechanical thrombectomy systems 10, 200. However, this is not intended to limit the disclosure as the discussion may be applied to other similar members and/or components of members or systems disclosed herein.

The various components of the mechanical thrombectomy systems 10, 200 may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the various components of the mechanical thrombectomy systems 10, 200 may also be doped with, made of, or otherwise include a radiopaque material including those listed herein or other suitable radiopaque materials. In some embodiments, a degree of MRI compatibility is imparted into the mechanical thrombectomy systems 10, 200. For example, to enhance compatibility with Magnetic Resonance Imaging (MRI) machines, it may be desirable to make the various components of the mechanical thrombectomy systems 10, 200, in a manner that would impart a degree of MRI compatibility. For example, the various components of the mechanical thrombectomy systems 10, 200, may be made of a material that does not substantially distort the image and create substantial artifacts (artifacts are gaps in the image). Certain ferromagnetic materials, for example, may not be suitable because they may create artifacts in an MRI image. The various components of the mechanical thrombectomy systems 10, 200, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers that may be used to form the various components of the mechanical thrombectomy systems 10, 200 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the mechanical thrombectomy systems 10, 200 may include a coating, for example a lubricious, a hydrophilic, a protective, or other type of coating. Hydrophobic coatings such as fluoropolymers provide a dry lubricity which improves device handling and exchanges. Lubricious coatings improve steerability and improve lesion crossing capability. Suitable lubricious polymers may include silicone and the like, polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

It should be understood that this disclosure is, in many respects, only illustrative. Changes may be made in details, particularly in matters of shape, size, and arrangement of steps without exceeding the scope of the disclosure. The disclosure's scope is, of course, defined in the language in which the appended claims are expressed.

What is claimed is:

1. A mechanical thrombectomy system, the system comprising:
an outer catheter extending from a proximal end to a distal end and defining a first lumen extending therethrough;
an inner sheath extending from a proximal end to a distal end and defining a second lumen extending therethrough, the inner sheath slidably disposed within the first lumen of the outer catheter;
an inner shaft extending from a proximal end to a distal end region, the inner shaft slidably disposed within the second lumen of the inner sheath;
an expandable anchor mechanism coupled to the distal end region of the inner shaft, the expandable anchor mechanism configured to move between a collapsed delivery configuration and an expanded configuration; and
a handle coupled to the proximal end of the outer catheter.

2. The mechanical thrombectomy system of claim 1, wherein the anchor mechanism comprises a torsion spring.

3. The mechanical thrombectomy system of claim 2, wherein the torsion spring comprises a helically wound portion and one or more free ends.

4. The mechanical thrombectomy system of claim 3, wherein the helically wound portion biases the one or more free ends into an expanded configuration.

5. The mechanical thrombectomy system claim 3, wherein a biasing force is configured to radially deflect the one or more free ends radially inwards.

6. The mechanical thrombectomy system of claim 3, further comprising a polymer jacket disposed over at least a portion of the one or more free ends.

7. The mechanical thrombectomy system of claim 1, wherein the anchor mechanism comprises an expandable basket.

8. The mechanical thrombectomy system of claim 7, wherein the expandable basket is self-expanding.

9. The mechanical thrombectomy system of claim 7, further comprising a central wire coupled to a distal end of the expandable basket.

10. The mechanical thrombectomy system claim 1, further comprising an outer sheath extending from a proximal end to a distal end, the outer sheath disposed over the outer catheter.

11. The mechanical thrombectomy system of claim 10, wherein the outer catheter further comprises an expandable distal end region adjacent to the distal end, the expandable distal end region movable between a collapsed configuration and an expanded configuration.

12. The mechanical thrombectomy system of claim 11, wherein the expandable distal end region comprises two or more support members and a membrane extending between the two or more support members.

13. The mechanical thrombectomy system of claim 11, wherein when the outer sheath is disposed over the expandable distal end region, the expandable distal end region is biased into the collapsed configuration and when the outer sheath is proximally retracted relative to the outer catheter, the expandable distal end region expands into the expanded configuration.

14. The mechanical thrombectomy system of claim 1, wherein when the inner sheath is disposed over the anchor mechanism, the anchor mechanism is biased into the collapsed configuration and when the inner sheath is proximally retracted relative to the anchor mechanism, the anchor mechanism expands into the expanded configuration.

15. The mechanical thrombectomy system of claim 14, wherein when the anchor mechanism is in the expanded configuration, distal advancement of the inner sheath biases the anchor mechanism to the collapsed configuration.

16. A mechanical thrombectomy system, the system comprising:
an outer sheath extending from a proximal end to a distal end and defining a first lumen extending therethrough;
an intermediate sheath extending from a proximal end to a distal end and defining a second lumen extending therethrough, the intermediate sheath slidably disposed within the first lumen of the outer sheath;
an inner sheath extending from a proximal end to a distal end and defining a third lumen extending therethrough, the inner sheath slidably disposed within the second lumen of the intermediate sheath;
an inner shaft extending from a proximal end to a distal end region, the inner shaft slidably disposed within the second lumen of the inner sheath; and
an expandable anchor mechanism coupled to the distal end region of the inner shaft, the expandable anchor mechanism configured to move between a collapsed delivery configuration and an expanded configuration, the anchor mechanism comprising a torsion spring.

17. The mechanical thrombectomy system of claim 16, wherein the intermediate sheath further comprises an expandable distal end region adjacent to the distal end, the expandable distal end region movable between a collapsed configuration and an expanded configuration.

18. The mechanical thrombectomy system of claim 17, wherein the expandable distal end region comprises two or more support members and a membrane extending between the two or more support members.

19. The mechanical thrombectomy system of claim 17, wherein when the outer sheath is disposed over the expandable distal end region, the expandable distal end region is biased into the collapsed configuration and when the outer sheath is proximally retracted relative to the intermediate sheath, the expandable distal end region expands into the expanded configuration.

20. A method for removing a clot, the method comprising:
advancing a mechanical thrombectomy system through a vasculature to a target location, the mechanical thrombectomy system comprising:
an outer catheter extending from a proximal end to a distal end and defining a first lumen extending therethrough;
an inner sheath extending from a proximal end to a distal end and defining a second lumen extending therethrough, the inner sheath slidably disposed within the first lumen of the outer catheter;
an inner shaft extending from a proximal end to a distal end region, the inner shaft slidably disposed within the second lumen of the inner sheath; and
an expandable anchor mechanism coupled to the distal end region of the inner shaft, the expandable anchor mechanism configured to move between a collapsed delivery configuration and an expanded configuration;
distally advancing the inner sheath and the inner shaft through a clot in the vasculature;
proximally retracting the inner sheath to expand the anchor mechanism;
distally advancing the outer catheter through the clot; and
distally advancing the inner sheath or the outer catheter to recapture the anchor mechanism.
